(19) United States
(12) Patent Application Publication
(10) Pub. No.: US 2024/0189580 A1
(43) Pub. Date: Jun. 13, 2024

(54) STIMULATION LEADS AND SYSTEMS FOR CANCER TREATMENT WITH LOW RESISTANCE AND HEAT GENERATION

(71) Applicant: Cardiac Pacemakers, Inc., St. Paul, MN (US)

(72) Inventors: Devon N. Arnholt, Shoreview, MN (US); Michael J. Kane, St. Paul, MN (US); Brian L. Schmidt, White Bear Lake, MN (US)

(73) Assignee: MAYO FOUNDATION FOR MEDICAL EDUCATION AND RESEARCH, Rochester, MN (US)

(21) Appl. No.: 18/530,935

(22) Filed: Dec. 6, 2023

Related U.S. Application Data
(60) Provisional application No. 63/430,749, filed on Dec. 7, 2022.

(51) Int. Cl. A61N 1/05; A61N 1/40

(52) U.S. Cl. CPC A61N 1/0526; A61N 1/40

(57) ABSTRACT

Embodiments herein relate to implantable cancer therapy systems and stimulation leads thereof with low resistance. In an embodiment, an implantable lead for a cancer treatment system is included having a lead body with a proximal end and a distal end and one or more electric field generating electrodes. The electrodes are disposed along a length of the lead body. A plurality of electrical wires are included and disposed within the lead body. The plurality of electrical wires provides an electrical connection between at least one of the electrodes and the proximal end of the lead body. At least two of the plurality of electrical wires are arranged in parallel and connected to the same electrode. Other embodiments are also included herein.

This application claims the benefit of U.S. Provisional Application No. 63/430,749, filed Dec. 7, 2022, the content of which is herein incorporated by reference in its entirety.

### FIELD

Embodiments herein relate to implantable cancer therapy systems and stimulation leads thereof with low resistance.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy. Additional second-line therapies can include radioactive seeding, cryotherapy, hormone or biologics therapy, ablation, and the like. Combinations of first-line therapies and second-line therapies can also be a benefit to patients if one particular therapy on its own is not effective.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Various cancer therapies may have significant side effects on heathy tissue. Such side effects can vary widely depending on the type of cancer therapy but can manifest as anemia, thrombocytopenia, edema, alopecia, infections, neutropenia, lymphedema, cognitive problems, nausea and vomiting, neuropathy, skin and nail changes, sleep problems, urinary and bladder problems, and the like.

### SUMMARY

Embodiments herein relate to implantable cancer therapy systems and stimulation leads thereof with low resistance. In a first aspect, an implantable lead for a cancer treatment system can be included having a lead body with a proximal end, a distal end, and one or more electric field generating electrodes disposed along a length of the lead body. A plurality of electrical wires can be disposed within the lead body. The plurality of electrical wires can provide an electrical connection between at least one of the electrodes and the proximal end of the lead body. At least two of the plurality of electrical wires can be arranged in parallel and connected to the same electrode.

In a second aspect, in addition to one or more of the preceding or following aspects, or in the alternative to some aspects, the electrodes can include a proximal end, and a distal end and the plurality of electrical wires arranged in parallel can be connected to a middle portion of the electrodes between the proximal end and the distal end.

In a third aspect, in addition to one or more of the preceding or following aspects, or in the alternative to some aspects, a set of the plurality of electrical wires reflecting all wires connected to a particular electrode of the one or more electric field generating electrodes have a resistance of less than 0.4 ohm/cm.

In a fourth aspect, in addition to one or more of the preceding or following aspects, or in the alternative to some aspects, a set of the plurality of electrical wires reflecting all wires connected to a particular electrode of the one or more electric field generating electrodes have a resistance of less than or equal to 0.2 ohm/cm.

In a fifth aspect, in addition to one or more of the preceding or following aspects, or in the alternative to some aspects, the implantable lead for a cancer treatment system can further include a connection plug. The connection plug can include a plurality of electrical terminals. The connection plug can be disposed at the proximal end of the lead body and can be in electrical communication with the plurality of electrical wires.

In a sixth aspect, in addition to one or more of the preceding or following aspects, or in the alternative to some aspects, the resistance of the electrical wires arranged in parallel and connected to the same electrode as measured from an electrical terminal to a point of connection with the electrode can be less than 5 ohms.

In a seventh aspect, in addition to one or more of the preceding or following aspects, or in the alternative to some aspects, the resistance of the electrical wires arranged in parallel and connected to the same electrode as measured from an electrical terminal to a point of connection with the electrode can be less than or equal to 3.5 ohms.

In an eighth aspect, in addition to one or more of the preceding or following aspects, or in the alternative to some aspects, the lead body further defines a lumen, wherein the plurality of electrical wires can be disposed within the lumen.

In a ninth aspect, in addition to one or more of the preceding or following aspects, or in the alternative to some aspects, the electrodes can be disposed circumferentially around the lead body and extend a distance along a longitudinal axis of the lead body.

In a tenth aspect, in addition to one or more of the preceding or following aspects, or in the alternative to some aspects, at least three of the plurality of electrical wires can be arranged in parallel and connected to the same electrode.

In an eleventh aspect, in addition to one or more of the preceding or following aspects, or in the alternative to some aspects, the plurality of electrical wires have a diameter of less than 130 microns.

In a twelfth aspect, in addition to one or more of the preceding or following aspects, or in the alternative to some aspects, the electrodes can include a flat ribbon of metal, wherein the flat ribbon of metal can be wrapped around the lead body.

In a thirteenth aspect, in addition to one or more of the preceding or following aspects, or in the alternative to some aspects, the electrodes can include at least two electrical field generating electrodes, wherein the at least two electrical field generating electrodes can be separated by a distance along a longitudinal axis of the lead body.

In a fourteenth aspect, an implantable lead for a cancer treatment system can be included having a lead body with a proximal end and a distal end. The lead can also have one or more electric field generating electrodes. The electrodes can include a plurality of discrete segments, wherein the plurality of discrete segments can be electrically isolated from direct connection with one another. The electrodes can be disposed along a length of the lead body. A plurality of electrical wires can be disposed within the lead body. The plurality of electrical wires can provide an electrical connection between the electrodes and the proximal end of the lead body. The plurality of electrical wires can be separately connected to the discrete segments of the electrodes.

In a fifteenth aspect, in addition to one or more of the preceding or following aspects, or in the alternative to some aspects, the discrete segments can include three to ten segments.

In a sixteenth aspect, in addition to one or more of the preceding or following aspects, or in the alternative to some aspects, the plurality of discrete segments can have a length along the longitudinal axis of the lead body from 0.25 to 70 mm.

In a seventeenth aspect, in addition to one or more of the preceding or following aspects, or in the alternative to some aspects, the plurality of discrete segments can wrap around the lead body circumferentially.

In an eighteenth aspect, in addition to one or more of the preceding or following aspects, or in the alternative to some aspects, the implantable lead can further include a temperature sensor, wherein the temperature sensor can be disposed between two adjacent discrete segments.

In a nineteenth aspect, an implantable cancer treatment system can be included having an implantable housing. The implantable housing can include an electric field generating circuit and control circuitry disposed therein. The control circuitry causes the electric field generating circuit to generate the one or more electric fields at frequencies selected from a range of between 10 kHz to 1 MHz within a bodily tissue. The one or more electric fields can be effective to prevent and/or disrupt cellular mitosis in a cancerous cell. The system can further include a therapy lead. The therapy lead can include a lead body. The lead body can include a proximal end and a distal end. The therapy lead can further include one or more electric field generating electrodes. The electrodes can include a plurality of discrete segments. The plurality of discrete segments can be electrically isolated from direct connection with one another. The electrodes can be disposed along a length of the lead body. A plurality of electrical wires can be disposed within the lead body. The plurality of electrical wires provide an electrical connection between the electrodes and the proximal end of the lead body. The plurality of electrical wires can be separately connected to the discrete segments of the electrodes. The implantable cancer treatment system can be configured to execute a duty cycle scheme wherein electrical fields can be delivered using one or more individual discrete segments during a first phase of a cycle alternating with a separate one or more individual discrete segments during a second phase of the cycle.

In a twentieth aspect, in addition to one or more of the preceding or following aspects, or in the alternative to some aspects, the discrete segments can include two to ten segments.

In a twenty-first aspect, in addition to one or more of the preceding or following aspects, or in the alternative to some aspects, the plurality of discrete segments can have a length along the longitudinal axis of the lead body from 0.25 to 70 mm.

In a twenty-second aspect, in addition to one or more of the preceding or following aspects, or in the alternative to some aspects, the plurality of discrete segments can wrap around the lead body circumferentially.

In a twenty-third aspect, in addition to one or more of the preceding or following aspects, or in the alternative to some aspects, the therapy lead can further include a temperature sensor, wherein the temperature sensor can be disposed between two adjacent discrete segments.

In a twenty-fourth aspect, in addition to one or more of the preceding or following aspects, or in the alternative to some aspects, the duty cycle scheme can be modulated based on a signal from the temperature sensor.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a medical device implanted in a patient in accordance with various embodiments herein.

FIG. 2 is a schematic view of a placement of various cancer therapy stimulation leads in a region of a tumor resection site in accordance with various embodiments herein.

FIG. 3 is a schematic view of a cancer therapy stimulation lead in accordance with various embodiments herein.

FIG. 4 is a cross-sectional view of the lead in FIG. 3 along line 4-4′ in accordance with various embodiments herein.

FIG. 5 is a perspective view of a cancer therapy stimulation lead in accordance with various embodiments herein.

FIG. 6 is a perspective view of a deconstructed cancer therapy stimulation lead in accordance with various embodiments herein.

FIG. 7 is a cross-sectional schematic view of a portion of a lead including an electrode in accordance with various embodiments herein.

FIG. 8 is a cross-sectional schematic view of a portion of a lead including an electrode in accordance with various embodiments herein.

FIG. 9 is a cross-sectional schematic view of a portion of a lead including an electrode in accordance with various embodiments herein.

FIG. 10 is a cross-sectional schematic view of a portion of a lead including an electrode in accordance with various embodiments herein.

FIG. 11 is a cross-sectional schematic view of a portion of a lead including an electrode in accordance with various embodiments herein.

FIG. 12 is a cross-sectional schematic view of a portion of a lead including an electrode in accordance with various embodiments herein.

FIG. 13 is a cross-sectional schematic view of a portion of a lead including an electrode in accordance with various embodiments herein.

FIG. 14 is a cross-sectional schematic view of a portion of a lead including an electrode in accordance with various embodiments herein.

FIG. 15 is a cross-sectional schematic view of a portion of a lead including an electrode in accordance with various embodiments herein.

FIG. 16 is a cross-sectional schematic view of a portion of a lead including an electrode in accordance with various embodiments herein.

FIG. 17 is a cross-sectional schematic view of a portion of a lead including an electrode in accordance with various embodiments herein.

FIG. 18 is a cross-sectional schematic view of a portion of a lead including an electrode in accordance with various embodiments herein.

FIG. 19 is a cross-sectional schematic view of a portion of a lead including an electrode in accordance with various embodiments herein.

FIG. 20 is a cross-sectional schematic view of a portion of a lead including an electrode in accordance with various embodiments herein.

FIG. 21 is a schematic cross-sectional view of a medical device in accordance with various embodiments herein.

FIG. 22 is a schematic diagram of components of a medical device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Implantable medical devices that can provide electrical stimulation therapy are provided. In some embodiments, the electrical stimulation therapy can be used to treat cancer and/or tumor resection boundaries. However, electrical stimulation can generate heat and healthy tissue can be damaged if exposed to too much heat.

Various embodiments provided herein can include electrical stimulation leads and/or electrodes disposed thereon with a reduced resistance. The reduced resistance can result in less heat generation through Joule heating and/or other mechanisms. Therefore, lead designs herein can reduce the amount of heat generated for a given power level of electrical stimulation. As such, embodiments of implantable medical devices and/or electrical stimulation leads herein can be advantageous to provide electrical stimulation therapy at desired intensity levels (as a function of field strength, duty cycles, etc.) while resulting in less heat generation and therefore minimizing any adverse effects on healthy tissue.

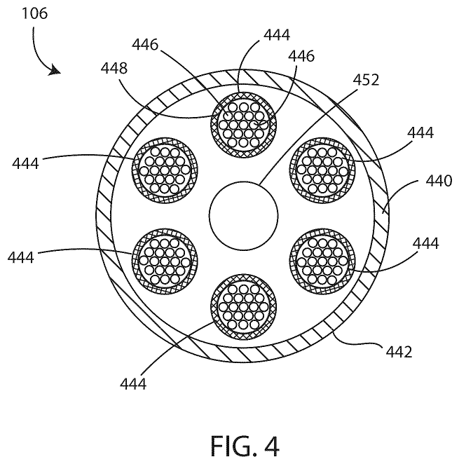

While not intending to be bound by theory, the use of a large number of conductors (as may take the form of wires aggregated into a plurality of cables) to provide electrical communication between electrodes on electrical stimulation leads and the proximal portions or ends thereof can effectively reduce the resistance (and/or impedance) of the electrical stimulation leads and/or components thereof in a substantial manner. Further, in some embodiments, connections between conductors and electrodes can be configured to reduce resistance and/or impedance. Examples of such configurations are shown with respect to FIG. 4 below and in other places herein.

As such, electrical stimulation leads and/or components thereof can exhibit reduced resistance (and/or impedance) versus otherwise similar electrical stimulation leads and/or components lacking features described herein. For example, resistance (and/or impedance) can be reduced by about 10, 20, 30, 40, 50, 60, 70, 80, or 90 percent or more, or by an amount falling within a range between any of the foregoing.

Resistance reduction of the conductor portion herein associated with the use of parallel conductors can be described by the equation K*ohms/N, where N is the number of equivalent parallel conductors and K is a constant describing our conductor resistivity relative to the reference conductor resistivity. In some embodiments, the resistance of the conductors arranged in parallel and connected to the same electrode as measured from an electrical terminal to a point of connection with the electrode is less than 7.5, 5, 4, 3.5, 3, 2.5, or 2 ohms.

In various embodiments, the resistance of the electrical stimulation lead can be less than 25 ohms, 15 ohms, 12 ohms, 10 ohms, 9 ohms, 8 ohms, 7 ohms, or 6 ohms. In some embodiments that include two cables per electrode (with each cable representing a bundle of discrete wires), the lead can have a resistance of about 0.2 ohms/cm. In some embodiments that include three cables per electrode, the lead can include a resistance of about 0.13 ohms/cm. In some embodiments that include 4 cables per electrode, the lead can have a resistance of about 0.1 ohms/cm. In contrast, otherwise similar leads that have one cable per electrode can have a resistance of about 0.4 ohms/cm. In some embodiments, a set of the plurality of electrical wires reflecting all wires connected to a particular electrode of the one or more electric field generating electrodes can have a resistance of less than 0.4 ohm/cm, 0.3 ohm/cm, 0.2 ohm/cm, or 0.1 ohm/cm, or an amount falling within a range between any of the foregoing. It will be appreciated that references herein to resistance shall also include impedance as relevant for AC unless the context dictates otherwise.

In various embodiments, as measured from the terminal of the lead to the point of attachment to the electrode (assuming a distance of 30 centimeters as an example and recognizing that resistance varies proportionally to length assuming no other changes are made), the resistance can be less than or equal to 7.5, 5.0, 4.5, 4.0, 3.5, 3.0, 2.5, or 2.0 ohms, or an amount falling within a range between any of the foregoing.

Various embodiments herein can also include additional resistance improvements based on electrode configurations and electrode/conductor interface configurations. For example, attaching the conductor(s) to the middle of the electrode instead of to an end can reduce resistance.

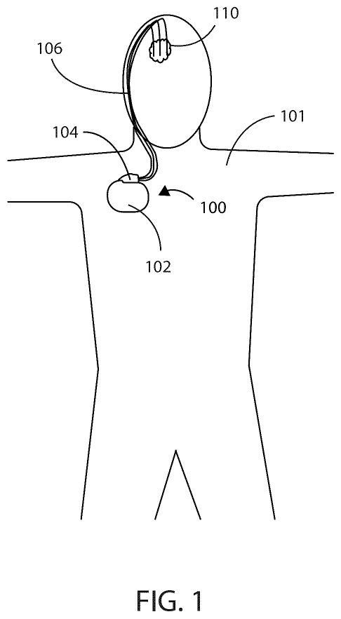

Referring now to FIG. 1, a schematic view of a medical device 100 implanted in a patient 101 is shown in accordance with the embodiments herein. In FIG. 1, the patient 101 has the medical device 100 implanted entirely within their body at or near a tumor resection site 110. It will be appreciated that while many embodiments herein disclose a tumor resection site 110, the area being treated may alternatively represent a cancerous or non-cancerous tumor site, zone, or cavity. Various implant sites can be used including areas such as in the limbs, the upper torso, the abdominal area, the head, and the like. In some embodiments, the medical device 100 can be at least partially implanted within the body of the patient at or near the site of the cancerous tumor. In various embodiments, the medical device can be entirely external to the patient. In some embodiments, the medical device can be partially external to the patient. In some embodiments, the medical device can be partially implanted and partially external to the body of a patient. In other embodiments, a partially implanted medical device can include a transcutaneous connection between components disposed internal to the body and external to the body. A partially or fully implanted medical device can wirelessly communicate with a partially or fully external portion of a medical device over a wireless connection.

In some embodiments, a portion of the medical device can be entirely implanted, and a portion of the medical device can be entirely external. For example, in some embodiments, one or more electrodes or leads can be entirely implanted within the body, whereas the portion of the medical device that generates an electric field, such as an electric field generator, can be entirely external to the body. It will be appreciated that in some embodiments described herein, the electric field generators described can include many of the same components and can be configured to perform many of the same functions, as a pulse generator. In embodiments where a portion of a medical device is entirely implanted, and a portion of the medical device is entirely external, the portion of the medical device that is entirely external can communicate wirelessly with the portion of the medical device that is entirely internal. However, in other embodiments a wired connection can be used.

In some embodiments, the portion of the medical device that is entirely implanted within the patient includes the field generation function and a wireless communication system. The wireless communication system can be configured to allow the medical device to communicate with an extracorporeal element of the system that provides the energy for the implanted device portions. The external portion can be integrated with a patient/clinician interface or the external portion can be in communication with a patient/clinician interface.

The medical device 100 can include a housing 102 and a header 104 coupled to the housing 102. Various materials can be used. However, in some embodiments, the housing 102 can be formed of a material such as a metal, ceramic, polymer, composite, or the like. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments where a portion of the medical device 100 is partially external, the header 104 and housing 102 can be surrounded by a protective casing made of durable polymeric material. In other embodiments, where a portion of the medical device 100 is partially external, the header 104 and housing 102 can be surrounded by a protective casing made of a combination of polymeric material, metallic material, and/or glass material.

Header 104 can be coupled to one or more leads, such as leads 106. The header 104 can serve to provide fixation of the proximal end of one or more leads 106 and electrically couple the one or more leads 106 to one or more components within the housing 102.

The one or more leads 106 can include one or more electrodes (not shown in this view) disposed along the length of the leads 106. In some embodiments, electrodes can include electric field generating electrodes, also referred to herein as “supply electrodes.” In some embodiments electrodes can include electric field sensing electrodes, also referred to herein as “sensing electrodes.” In some embodiments, leads 106 can include both supply electrodes and sensing electrodes. In other embodiments, leads 106 can include any number of electrodes that are both supply electrodes and sensing electrodes.

The one or more leads 106 can also include one or more temperature sensors (not shown in this view) disposed along the length of the leads 106. In some embodiments, a temperature sensor can be disposed between two adjacent discrete segments of an electrode. In some embodiments, a temperature sensor can be disposed under an electrode, such as between a lead body and the electrode. In some embodiments, a temperature sensor can be disposed adjacent to an electrode. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 106, temperature can be measured at a distance therefrom. Exemplary electrical temperature sensors can include, but are not limited to, thermistors, resistive temperature detectors, thermocouples, semiconductor based temperature sensors, and the like.

In some embodiments, the medical device system can further include a temperature sensor disposed remotely from the medical device. A remote temperature sensor can provide temperature data in addition to or in replace of temperature sensors in other areas such as along the leads 106. In some embodiments, a remote temperature sensor can be used to gather a core or reference temperature of the patient into which the system is implanted.

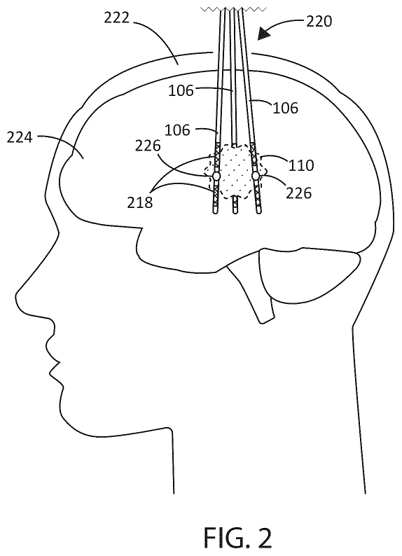

In some embodiments, the medical device 100 can include a plurality of stimulation leads implanted at or near a site a cancerous tumor or tumor resection. Referring now to FIG. 2, a schematic view of a placement of various cancer therapy stimulation leads 106 in a region of a tumor resection site 110 or tumor is shown in accordance with various embodiments herein. In the example of FIG. 2, temperature sensors 226 are disposed on the stimulation leads. The temperature sensors 226 can be of any type described elsewhere herein. In the embodiment shown in FIG. 2, each cancer therapy stimulation lead includes two electric field generating electrodes 218 disposed along a length of the cancer therapy stimulation leads. Each cancer therapy stimulation lead includes a proximal and a distal electric field generating electrode. It will be appreciated that each cancer therapy stimulation lead can include one or more electric field generating electrodes. Exemplary cancer therapy stimulation leads are disclosed in commonly owned U.S. Pat. Appl. No. 63/298,528, the content of which is hereby incorporated by reference in its entirety. The cancer therapy stimulation leads and the electric field generating electrodes disposed thereon are discussed in more detail below.

The side view shown in FIG. 2 also includes the placement of cancer therapy stimulation leads 106 around the tumor resection site 110 and in position within a burr hole 220 entry point on the patient's skull 222 within the patient's brain 224. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

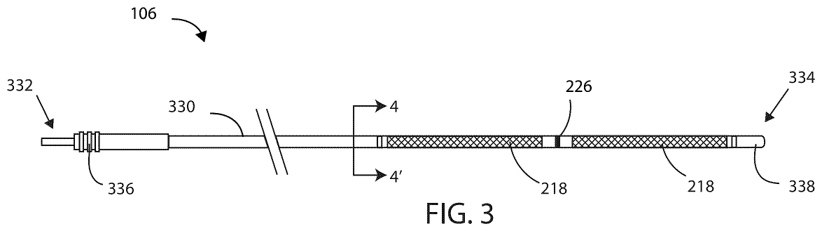

Referring now to FIG. 3, a schematic view of an exemplary cancer therapy stimulation lead 106 is shown in accordance with various embodiments herein. The cancer therapy stimulation lead 106 can include a lead body 330 with a proximal end 332 and a distal end 334. In this example, two electrodes 218 are coupled to the lead body 330, such as positioned near a distal end 334 thereof. In some embodiments, the electrodes 218 can include electric field generating electrodes. In various embodiments, the electrodes 218 can include electric field sensing electrodes. The electrodes 218 can be internally connected or internally independent. In an example where the electrodes 218 are independent, the system herein can model each as an independent field and heat source. The lead body 330 can define a lumen. The electrodes 218 can include various conductive materials such as platinum, silver, gold, iridium, titanium, and various alloys. In some embodiments, the cancer therapy stimulation lead 106 includes more than two electrodes.

The cancer therapy stimulation lead 106 can include one or more therapy zone temperature sensors disposed along a length of the cancer therapy stimulation lead. In this example, a therapy zone temperature sensor 226 is positioned between the first electrode 218 and the second electrode 218. The therapy zone temperature sensor 226 can include an optical or electrical thermal sensor. For example, the therapy zone temperature sensor can include a thermistor. The therapy zone temperature sensor 226 can be used to measure the thermal heating about the cancer therapy stimulation lead to provide feedback to a clinician about the local thermal heating zone around the lead and provide a tissue temperature of the treatment site to the medical device. In various embodiments, the therapy zone temperature sensor 226 can provide a tissue temperature at a site offset from a surface of the electrodes 218, 218. If a tissue temperature of a site offset from the electrodes 218, 218 is measured, the medical device can compensate for the offset when measuring or estimating the temperature of the tissue. In some embodiments, the therapy zone temperature sensor 226 can measure or estimate the reference or core body temperature of the patient when the therapy is turned off or paused. While not intending to be bound by theory, in some scenarios it can be easier to get an accurate measurement of a reference or core body temperature when therapy is turned off or paused. In some embodiments, therapy zone temperature sensor data can be recorded and relayed to the clinician, patient, care provider, and/or medical record system.

The cancer therapy stimulation lead 106 can further include a terminal pin 336 and/or connection plug for connecting the cancer therapy stimulation lead 106 to a medical device, such as a cancer treatment device. The terminal pin 336 and/or connection plug can be compatible with various standards for lead-header interface design including the DF-1, VS-1, IS-1, LV-1 and IS-4 standards, amongst other standards.

While a single terminal pin 336 is shown in FIG. 3, in various embodiments herein the cancer therapy stimulation lead 106 can branch into multiple terminal pins. For example, the cancer therapy stimulation lead 106 can include 2, 3, 4, or more terminal pins. In some embodiments, each terminal pin 336 can provide electrical communication with one or more cables (described below) and/or conductors passing through the cancer therapy stimulation lead 106. In some embodiments, each terminal pin 336 can provide electrical communication with one or more electric field generating electrodes 218. In some embodiments, each connection plug can include a plurality of electrical terminals or contacts to provide an electrical connection with the conductors of the lead. In some embodiments, individual electrical terminals can be connected to multiple conductors, such as multiple cables and/or wires. However, in some embodiments, individual electrical terminals are connected to individual conductors (such as separate cables and/or wires). For example, in some embodiments individual electrical terminals are connected to conductors on a 1 to 1 basis.

In some embodiments, the cancer therapy stimulation lead 106 can further include a fixation element 338, such as an element that can adhere to a portion of the subject's body to maintain the position of the cancer therapy stimulation lead 106 and/or the electrodes 218. In various embodiments, the fixation element 338 can be disposed along the distal end 334 of the cancer therapy stimulation lead 106. However, in some embodiments a fixation element 338 is omitted.

FIG. 4 shows a cross-sectional schematic view of a lead 106 as taken along line 4-4′ of FIG. 3. The lead 106 can include an outer layer 440 with an outer surface 442. The outer layer 440 can be flexible and can be configured to protect other components disposed within the lead 106. In some embodiments, the outer layer 440 can be circular in cross-section. In some embodiments, the outer layer 440 includes a dielectric material and/or an insulator. In some embodiments, the outer layer 440 can include various biocompatible materials such as polysiloxanes, polyethylenes, polyamides, polyurethane and the like.

In various embodiments, the lead 106 can include one or more cables 444 or wire units, such as a first cable and a second cable when the lead 106 includes two cables 444, or a first, second, third, fourth, fifth, and sixth cable when the lead 106 includes six cables 444 (shown in FIG. 4). In various embodiments, the cables 444 and/or the wires within each cable can be arranged in parallel, such that each electrode 218 can include a connection with a plurality of cables 444 where at least some of the cables 444 are arranged in parallel.

In some embodiments, the one or more cables 444 can be disposed within the lead 106. The cables 444 can each be configured to provide electrical communication between an electrode 218 and the proximal end 332 of the lead 106. In some embodiments, such as shown in FIG. 4, each cable 444 can include a plurality of wires 446. In some embodiments, each cable 444 can include at least 1, 2, 3, 5, 10, 15, or at least 19 wires. In some embodiments, each cable 444 can include 19 wires 446, as shown in FIG. 4. In some embodiments, each of the wires 446 within a cable 444 can be electrically isolated from each other, such as by being enclosed in an insulator.

The cables 444 and/or wires 446 therein can include various materials including copper, aluminum, silver, gold, and various alloys such as tantalum/platinum, MP35N and the like. An insulator 448 can surround the cable 444 and/or individual wires 446 therein. The insulator 448 can include various materials such as electrically insulating polymers, such as a layer of ETFE or another polymer. In some embodiments, each wire 446 can have a diameter of about 50 microns. The wires 446 can be twisted together to form a bundle of wires within the insulator 448. In some embodiments, the bundles of wires can have an overall diameter of about 100, 110, 120, 130, 140, or 150 microns, or a diameter falling within a range between any of the foregoing.

A separate cable or set of cables can be in communication with each electrode disposed along the lead. In some embodiments, each of the electrodes 218 can have one, two, three, or more individual cables 444 to electrically couple the electrode 218 to the proximal end 332 of the lead 106. However, in some embodiments, each of the electrodes 218 only connects to a single cable to electrically couple the electrode 218 to the proximal end 332 of the lead 106. In some embodiments, the cables 444 can be configured as a coil, helix, spiral or the like. In various embodiments, wires of a cable 444 can form a part of an electrical circuit by which the electric fields from the electric field generating circuit are delivered to the site of the cancerous tissue. It will be appreciated that many more cables than are shown in FIG. 4 can be included within embodiments herein as well as fewer cables than are shown in FIG. 4. The lead 106 can include 1, 2, 3, 4, 5, 6, 7, 8, 10, 15 or 20 or more cables, or any number of cables falling within a range between any of the foregoing.

Cables can be used in leads to prevent flex fatigue issues. In some embodiments, each cable can include at least 2, 4, 6, 8, 10, 12, 14, 16, 18, 20, 22, 24, or 26 wires, or an amount of wires falling within a range between any of the foregoing. In some embodiments not more than 20 wires, such as 19 wires. In various embodiments, each electrode can have three cables 444, and the temperature sensor 226 can have separate conductors (not shown in FIG. 4) connected to it.

In some embodiments, the lead 106 can include a central channel 452 or lumen. The central channel 452 can be configured for a guide wire, or other implanting device, to pass through, such as to aid in implanting the lead 106 and electrodes 218. In some cases, additional channels (not shown) are disposed within the lead 106.

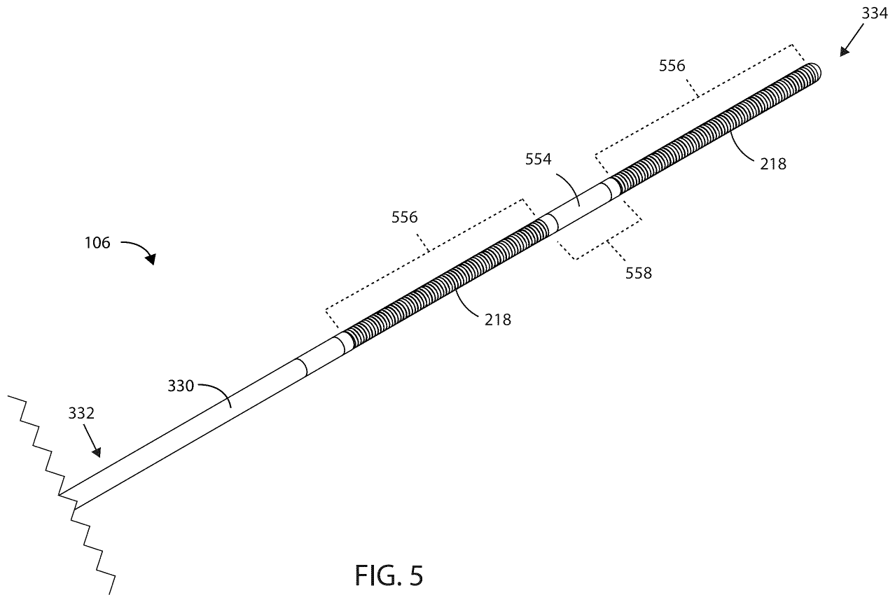

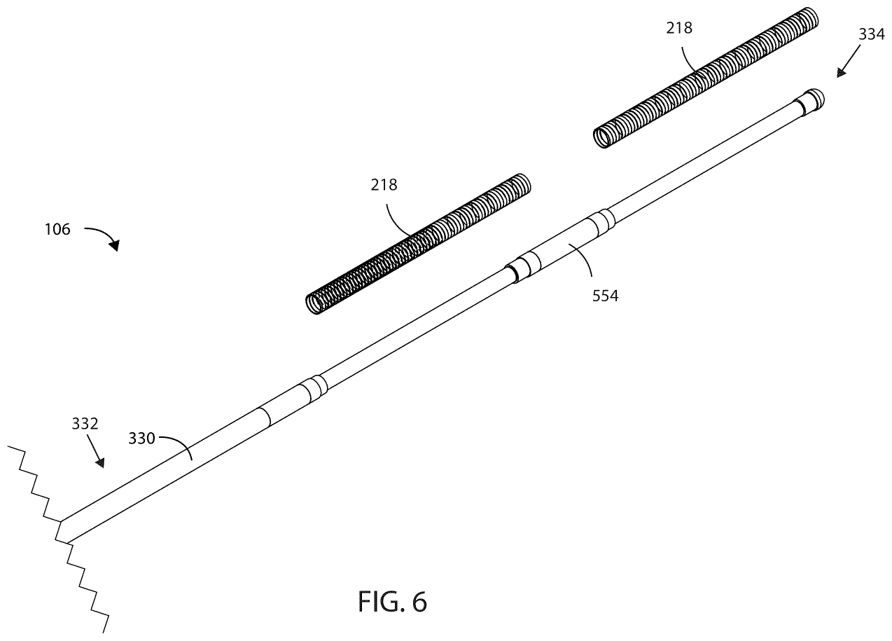

Referring now to FIG. 5 and FIG. 6, perspective views of lead 106 are shown in accordance with various embodiments. Leads 106 include one or more electrodes 218 disposed along a length of the lead body 330, where the electrodes 218 are separated by a non-conducting gap portion 554 on the exterior of the lead 106. The electrodes 218 can include an electrode length 556, and the non-conducting portion can include a non-conducting portion length of 558.

The electrode lengths 556 can each independently be from 1 cm to 4 cm in length. In some embodiments, the electrode length can be greater than or equal to 1.0 cm, 1.5 cm, 2.0 cm, 2.5 cm, 3.0 cm, 3.5 cm, 4.0 cm, 4.5 cm, 5.0 cm, 5.5 cm, or 6.0 cm, or can be an amount falling within a range between any of the foregoing. In various embodiments, when more than one electrode 218 is present along a length of the lead body, each electrode can be the same length. In various other embodiments, when more than one electrode 218 is present along a length of the lead body, each electrode can be a different length.

For leads including two or more electrodes 218, the non-conducting gap portion length 558 can be from 0.5 cm to 2 cm. In some embodiments, the non-conducing gap portion length can be greater than or equal to 0.25 cm, 0.50 cm, 0.75 cm, 1.00 cm, 1.25 cm, 1.50 cm, 1.75 cm, 2.00 cm, 2.25 cm, 2.5 cm, 2.75 cm, or 3 cm, or can be an amount falling within a range between any of the foregoing. In various embodiments, when more than one electrode 218 is present along a length of the lead body, each electrode can be separated by a non-conducting gap portion having the same length. In various other embodiments, when more than one electrode 218 is present along a length of the lead body, each electrode can be separated by a non-conducting gap portion having a different length.

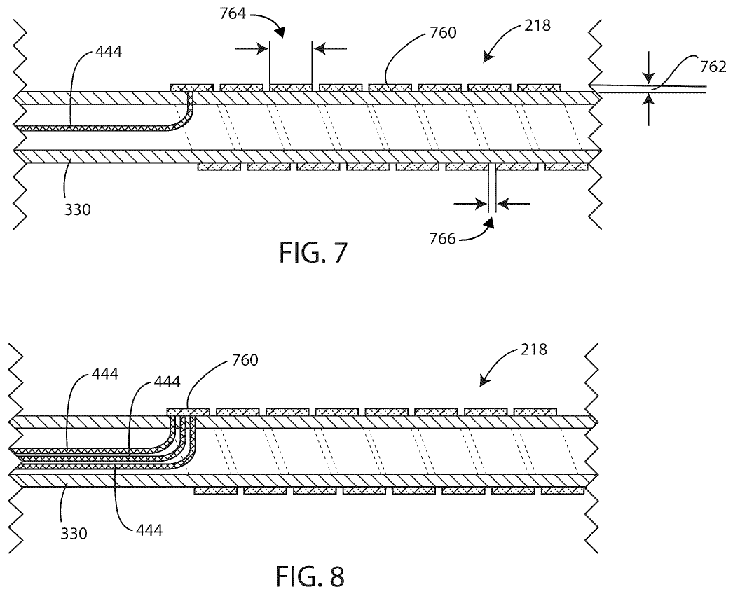

As discussed elsewhere herein, electrodes can include electric field generating electrodes. In various embodiments, the electrodes can be formed from a cylindrical wire, a ribbon wire, a walled tube, a sputtered metallic electrode, or the like as will be discussed in reference to FIGS. 7-20, which show cross-sectional views of an electrode 218 of the various embodied herein.

It should be noted that the electrodes 218 can refer to distal most electrodes, proximal most electrodes, or any electrodes 218 disposed therebetween. In various embodiments, the electrodes herein can be disposed about the entirety of the lead body. In other embodiments, the electrodes herein can be disposed partially about the lead body, so as to only generate an electric field about a portion of the lead body. Electrodes 218 that are not the distal most can include one or more additional cables extending through the lead body 330 and past the electrode 218, such as to electrically couple the more distal electrodes 218 to the proximal end 332 of the lead 106. In various embodiments, the electrodes herein can include those constructed of a metallic layer deposited as a sputter coating on a non-metallic substrate via a sputter coating process to achieve the thicknesses as discussed herein.

FIGS. 7-20 show cross-sectional views of various embodiments of electrodes 218 and cables 444 herein. FIG. 7 shows an electrode 218 including a ribbon wire or flat wire disposed helically or wrapped around the lead body 330. The cable 444 is electrically connected to the proximal portion of the electrode 218. In various embodiments, the electrode 218 can include a ribbon wire having a plurality of conductive ribbon segments 760 (in cross-section) disposed about the lead body 330. As shown in FIG. 7, the plurality of conductive ribbon segments 760 can be in electrical communication with cable 444 and disposed helically about lead body 330.

In various embodiments, when the ribbon wires are disposed circumferentially about the lead body 330, they form a circumferential conductor pattern. In various embodiments, when the ribbon wires are disposed helically about the lead body 330, they form a helical conductor pattern. The circumferential conductor pattern or helical conductor pattern can have an outside diameter of from 1 millimeter (mm) to 3 mm. In some embodiments, the outside diameter can be greater than or equal to 1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, or 3.0 mm, or can be an amount falling within a range between any of the foregoing.

The ribbon segments 760 can have a ribbon segment thickness 762 (or a thickness in a radial direction with respect to the lead body) and a ribbon segment width 764. While not intending to be bound by theory, the thickness 762 directly impacts the size of an MRI or imaging artifact. As such, in various embodiments, the thickness 762 can be sufficiently small to avoid significantly sized MRI or imaging artifacts allowing for a better view of the tissue and/or cancerous tumor within the patient.

In various embodiments, the ribbon segment thickness 762 can be greater than or equal to 0.00001 inches to 0.005 inches. In some embodiments, the ribbon segment thickness 762 can be greater than or equal to 0.00001 inches, 0.00005 inches, 0.0001 inches, 0.0005 inches, 0.001 inches, 0.005 inches, or can be an amount falling within a range between any of the foregoing. The ribbon segment width 764 can be greater than or equal to 0.00001 inches to 0.005 inches. In some embodiments, the ribbon segment width can be greater than or equal to 0.00001 inches, 0.00005 inches, 0.0001 inches, 0.0005 inches, 0.001 inches, 0.005 inches, 0.010 inches, 0.011 inches, 0.012 inches, 0.013 inches, 0.014 inches, or 0.015 inches, or can be an amount falling within a range between any of the foregoing. It will be appreciated that the ribbon wire thickness can extend in a radial direction with respect to the lead body and the ribbon wire width can extend in a longitudinal or axial direction with respect to the lead body.

The length of an entire electrode 218, such as the electrode length 556 shown in FIG. 5, created with ribbon wires can be greater than or equal to 0.5 centimeters (cm) to 5 cm. In some embodiments, the length of the entire electrode 218 can be greater than or equal to 0.5 cm, 1.0 cm, 1.5 cm, 2.0 cm, 2.5 cm, 3.0 cm, 3.5 cm, 4.0 cm, 4.5 cm, or 5.0 cm, or can be an amount falling within a range between any of the foregoing. In some embodiments, the length can be from 1 cm to 3 cm. In some embodiments, the electrodes 218 created with ribbon wires can have a length of about 2 cm.

An electrode ribbon wire or filar with both a small thickness and width may not have sufficient durability or structural integrity to work effectively as a supply or stimulation electrode. As such, in some embodiments, the ribbon wire or filar can have a relatively small thickness along with a larger width. As such, the total cross-sectional size of the ribbon wire or filar can still be sufficiently large so as to offer sufficient durability and/or structural integrity. In various embodiments, the ribbon wire can be sized to include an aspect ratio between a thickness and a width of the ribbon wire of from 2:4 to 2:20. In various embodiments, the ribbon wire can be sized to include an aspect ratio between a thickness and a width of the ribbon wire of from 2:4 to 2:8. In various embodiments, the ribbon wire can be sized to include an aspect ratio between a thickness and a width of the ribbon wire of from 2:10. In various embodiments where the electrodes include more than one ribbon wire, it will be appreciated that each individual ribbon wire can have an aspect ratio that is the same. In various embodiments where the electrodes include more than one ribbon wire, it will be appreciated that each individual ribbon wire can have an aspect ratio that is the different.

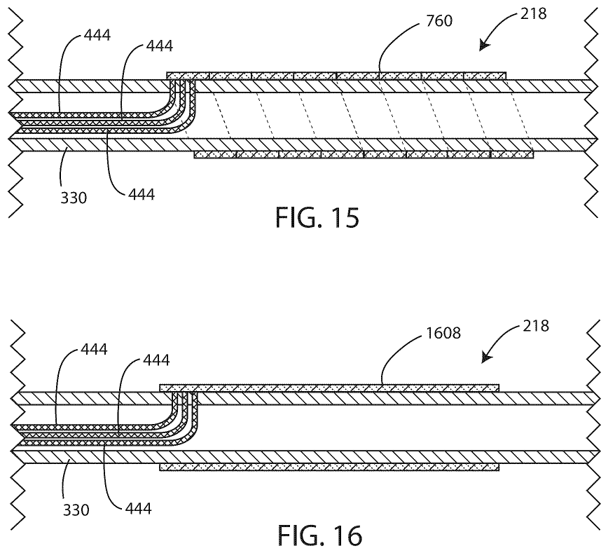

In some embodiments, the ribbon segments 760 can include a pitch 766 between successive or adjacent ribbon segments 760, such that adjacent ribbon segments do not contact each other along the lengthwise axis of the lead body 330. In some embodiments, the pitch 766 can be greater than or equal to width of the ribbon segments 760. In some embodiments, the pitch 766 can be greater than or equal to half the width of the ribbon segments 760. In some embodiments, the pitch 766 can be at least twice the width of the ribbon segments 760. In some embodiments, the pitch 766 can be at least three times the width of the ribbon segments 760. In some embodiments, the pitch 766 can be at least four times the width of the ribbon segments 760. In some embodiments, the pitch 766 can be at least five times the width of the ribbon segments 760. In some embodiments, the pitch 766 can be at least ten times the width of the ribbon segments 760. In various embodiments, the each of the ribbon segments 760 can contact neighboring ribbon segments on either side in a direction along the longitudinal axis, such as shown in FIG. 15.

In some embodiments, each electrode can include one or more cables 444 connected to it, such as shown in FIG. 7. In some embodiments, each electrode can include two, three, four, five, six, seven, eight, nine, or ten or more cables 444 connected to it.

In reference now to FIG. 8, a cross-sectional schematic view of a portion of a lead including an electrode 218 is shown in accordance with various embodiments herein. In some embodiments, the lead 106 can include a plurality of cables 444 electrically connecting the electrode 218 to a portion near or at the proximal end of the lead 106. In some embodiments, the lead 106 can include two cables 444, three cables 444, four cables 444, five cables 444 or more. In some embodiments, all of the cables 444 for an electrode 218 can be connected to a proximal portion of the electrode, such as a ⅓rd most proximal portion of the electrode 218 or a proximal most ribbon segment 760 (as shown in FIG. 8).

In other embodiments, all of the cables 444 for an electrode 218 can be connected to a distal portion of the electrode, such as a ⅓rd most distal portion of the electrode 218 or a distal most ribbon segment 760.

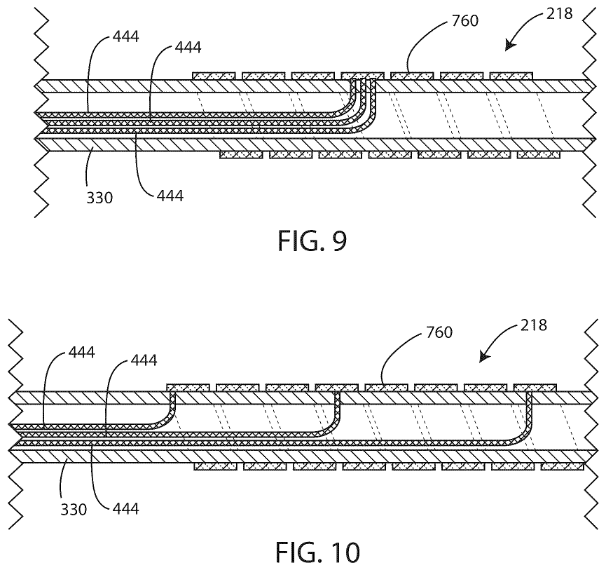

In other embodiments, cables 444 for an electrode 218 can be connected to a middle portion of the electrode, such as within the middle 25 percent of the length of the electrode 218 or a middle most ribbon segment 760 (as shown in FIG. 9). Connecting to the middle of the electrode instead of to only an end can reduce resistance.

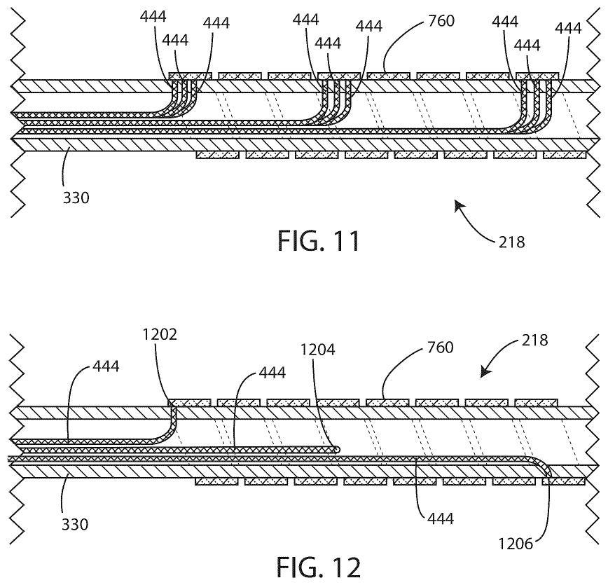

In still other embodiments, the cables 444 for an electrode 218 can be connected at different locations along the length of the electrode 218. In FIG. 10, one cable 444 is connected at a proximal portion of the electrode, one cable 444 is connected in a middle portion of the electrode, and one cable 444 is connected at a distal portion of the electrode. In FIG. 11, a plurality of cables 444 are connected at a proximal portion of the electrode, a plurality of cables 444 are connected in a middle portion of the electrode, and a plurality of cables 444 are connected at a distal portion of the electrode. In some embodiments, the connections between the cables 444 and the electrode can be distributed equally along the length. In some embodiments, the connections between the cables 444 and the electrode can be evenly distributed between a proximal portion and a distal portion.

In some embodiments, the connections between the cables 444 and electrode 218 can be aligned radially, such as shown in FIG. 11 where all of the connections are along the top side. However, in some embodiments, the connections between the cables 444 and the electrode 218 can be distributed radially, such as evenly distributed. FIG. 12 shows the connections between the cables 444 and the electrode 218 distributed radially. The first cable 444 is connected with the electrode 218 at location 1202. The second cable 444 is connected with the electrode 218 at a location 1204 (this cross-section does not actually show the connection at location 1204 as the cross-section is in a different plane than the location 1204). The third cable 444 is connected with the electrode 218 at location 1206. The location 1202 can be distributed 90 degrees away from the location 1204. The location 1204 can be distributed 90 degrees away from the location 1206. The location 1202 can be distributed 180 degrees away from the location 1206.

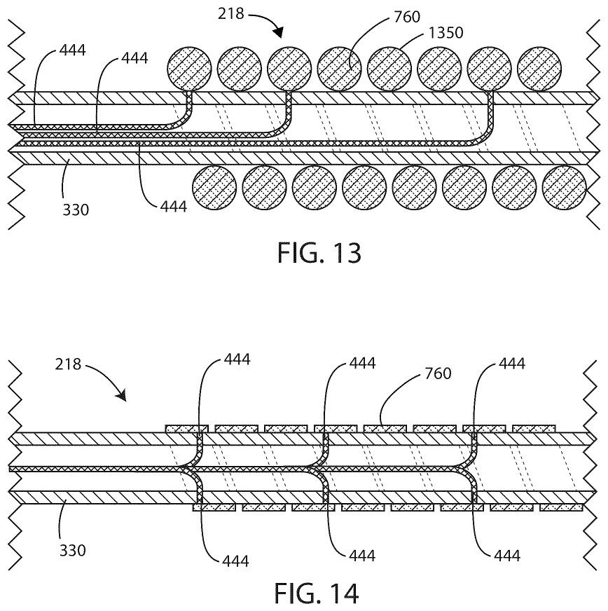

While FIGS. 7-12 illustrate the use of ribbon wire or flat wire for forming electrodes, it will be appreciated that many different cross-sectional shapes for materials or wires used to form the electrodes are contemplated herein. In reference now to FIG. 13, a cross-sectional schematic view of a portion of a lead including an electrode 218 is shown in accordance with various embodiments herein. The electrode 218 in FIG. 13 includes a wire 1350 with a substantially circular cross-section. In some embodiments, the electrode 218 can include a wire with a rectangular cross-section, such as shown in FIG. 7-12. In other embodiments, the electrode 218 has a cross-sectional shape of a square, an oval, a triangle, or an irregular shape.

In reference now to FIG. 14, a cross-sectional schematic view of a portion of a lead including an electrode 218 is shown in accordance with various embodiments herein. In some embodiments, the electrode 218 can include one or more cables 444 being connected to the electrode at the same location along the longitudinal axis of the lead body. In some embodiments, the cables can be connected to the electrode at the same longitudinal location, but at different radial locations, such as shown in FIG. 14.

In reference now to FIG. 15, a cross-sectional schematic view of a portion of a lead including an electrode 218 is shown in accordance with various embodiments herein. In this example, each of the ribbon segments 760 can contact neighboring ribbon segments on either side in a direction along the longitudinal axis, such as shown in FIG. 15. The electrode 108 of FIG. 15 does not include a pitch or gap between adjacent ribbon segments.

It will be appreciated that electrodes herein can take on many different forms. In reference now to FIG. 16, a cross-sectional schematic view of a portion of a lead including an electrode 218 is shown in accordance with various embodiments herein. In various embodiments, the electrode 108 can include a cylindrical electrode 1608. The cylindrical electrode 1608 can surround a portion of the lead body 330. The cylindrical electrode 1608 can be connected to one or more cables 444, as shown in the FIGS. 7-20.

In the embodiments shown in FIG. 16, the cylindrical electrode 1608 is connected to three cables 444. The three cables 444 are connected to a proximal end of the cylindrical electrode 1608. However, the cables 444 can be connected at other locations such as shown in FIGS. 7-20.

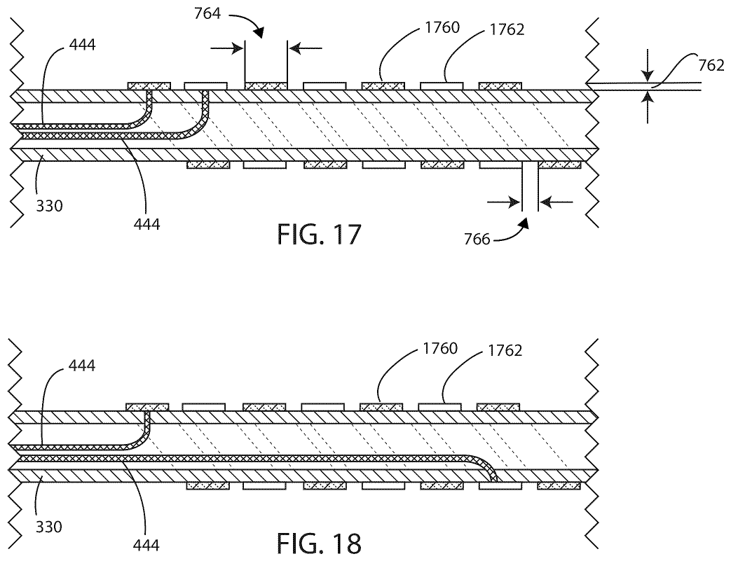

In reference now to FIGS. 17 and 18, cross-sectional schematic views of a portion of a lead including an electrode 218 are shown in accordance with various embodiments herein. In some embodiments, an electrode 218 can include a first ribbon portion 1760 and a second ribbon portion 1762. The electrode 218 can include alternating segments of the first ribbon portion 1760 and the second ribbon portion 1762 along the longitudinal axis of the lead body 330. In various embodiments, the first ribbon portion 1760 and the second ribbon portion 1762 can be disposed helically about the lead body 330. In various embodiments, a first cable 444 can be connected to the first ribbon portion 1760 and a second cable 444 can be connected to the second ribbon portion 1762. In some embodiments, the first ribbon portion 1760 can be electrically isolated from the second ribbon portion 1762 however in other embodiments they can be in electrical communication with one another.

In some embodiments, the ribbon segment thickness 762 and ribbon segment width 764 of the first ribbon portion 1760 and second ribbon segments 1762 can be the same. In some embodiments, the ribbon segment thickness 762 and ribbon segment width 764 of the first ribbon portion 1760 and second ribbon portion 1762 can be different. In some embodiments, such as shown in FIG. 17, the cables 444 can both be connected to proximal ends or proximal portions of the first ribbon portion 1760 and the second ribbon portion 1762. In some embodiments, such as shown in FIG. 18, the cables 444 can be connected to the ribbon portions 1760, 1762 at opposite ends. In some embodiments, a cable 444 can be connected to the first ribbon portion 1760 at a proximal end or a proximal portion. In some embodiments, a cable 444 can be connected to the second ribbon portion 1762 at a distal end or a distal portion.

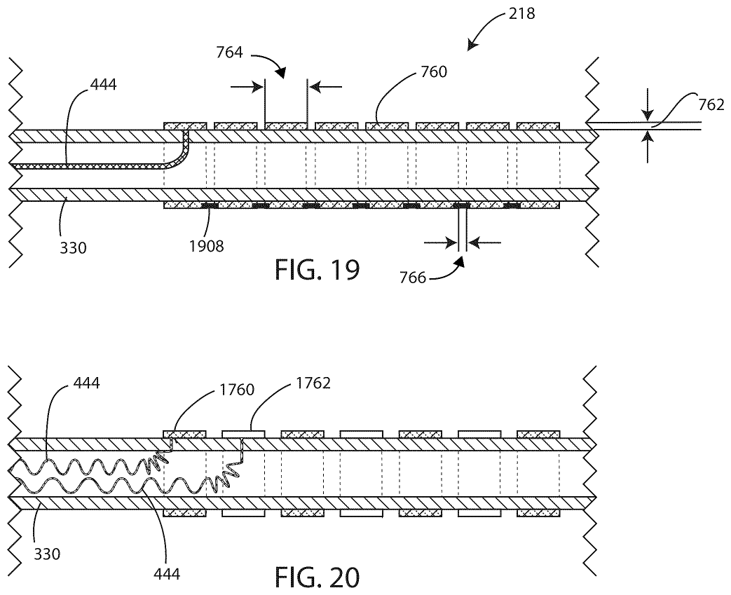

In some embodiments, the electrode 218 can include a plurality of conductive ribbon segments 760. In some embodiments, the ribbon segments 760 can be disposed circumferentially about the lead body 330, such as shown in FIGS. 19-20. In various embodiments, the ribbon segments 760 can each be an annular ring disposed around the lead body 330. Each ribbon segment 760 can be electrically connected to an adjacent ribbon segment 760 by a wire or other conductive elements 1908. In some embodiments, the conductive elements 1908 can be on the surface of the lead body 330, but in other embodiments the conductive elements 1908 can be embedded in or inside the lead body 330.

In reference now to FIG. 19, a cross-sectional schematic view of a portion of a lead including an electrode 218 is shown in accordance with various embodiments herein. As shown in FIG. 19, the plurality of conductive ribbon segments can include ribbon segments 760 that can be in electrical communication with cable 444 and disposed circumferentially about lead body 330.

In reference now to FIG. 20, a cross-sectional schematic view of a portion of a lead including an electrode 218 is shown in accordance with various embodiments herein. As shown in FIG. 20, the plurality of conductive ribbon segments can include first ribbon portion 1760 that can be in electrical communication with a cable 444 and second ribbon portion 1762 that can be in electrical communication with a different cable 444. Further the ribbon portions 1760, 1762 can include ribbon segments that can be disposed circumferentially about lead body 330. In some embodiments, segments can be electrically connected to other segments with wires or other conductive elements (not shown in this view).

In various embodiments, the cable 444 can be coiled or include a plurality of switch backs, such that the length of the cable 444 is greater than the distance between the proximal end of the lead and the portion of the electrode which the cable 444 is connected to. In some embodiments, the length of the cable 444 is at least 20%, 30%, 50%, 75%, or 100% longer than the distance between the proximal end of the lead and the portion of the electrode which the cable 444 is connected to.

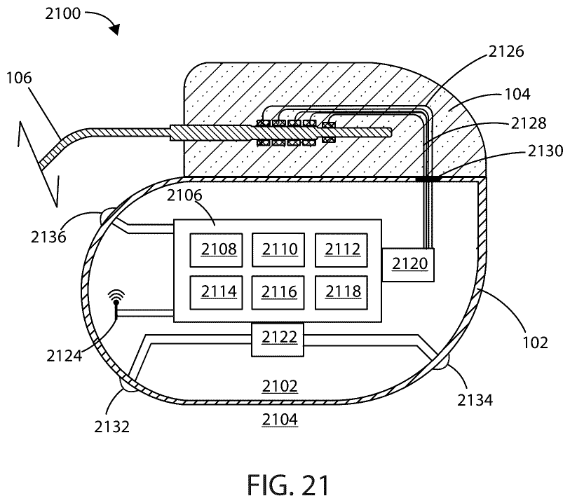

Referring now to FIG. 21, a schematic cross-sectional view of medical device 2100 is shown in accordance with various embodiments herein. The housing 102 can define an interior volume 2102 that can be hollow and that in some embodiments is hermetically sealed off from the area 2104 outside of medical device 2100. In other embodiments the housing 102 can be filled with components and/or structural materials such that it is non-hollow. The medical device 2100 can include control circuitry 2106, which can include various components 2108, 2110, 2112, 2114, 2116, and 2118 disposed within housing 102. In some embodiments, these components can be integrated and in other embodiments these components can be separate. In yet other embodiments, there can be a combination of both integrated and separate components. The medical device 2100 can also include an antenna 2124, to allow for unidirectional or bidirectional wireless data communication, such as with an external device or an external power supply. In some embodiments, the components of medical device 2100 can include an inductive energy receiver coil (not shown) communicatively coupled or attached thereto to facilitate transcutaneous recharging of the medical device via recharging circuitry.

The various components 2108, 2110, 2112, 2114, 2116, and 2118 of control circuitry 2106 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 2106 can be in communication with an electric field generating circuit 2120 that can be configured to generate electric current to create one or more fields. The electric field generating circuit 2120 can be integrated with the control circuitry 2106 or can be a separate component from control circuitry 2106. Control circuitry 2106 can be configured to control delivery of electric current from the electric field generating circuit 2120. In some embodiments, the electric field generating circuit 2120 can be present in a portion of the medical device that is external to the body.

In some embodiments, the control circuitry 2106 can be configured to direct the electric field generating circuit 2120 to deliver an electric field via leads 106 to the site of a cancerous tumor located within a bodily tissue. In other embodiments, the control circuitry 2106 can be configured to direct the electric field generating circuit 2120 to deliver an electric field via the housing 102 of medical device 2100 to the site of a cancerous tumor located within a bodily tissue. In other embodiments, the control circuitry 2106 can be configured to direct the electric field generating circuit 2120 to deliver an electric field between leads 106 and the housing 102 of medical device 2100. In some embodiments, one or more leads 106 can be in electrical communication with the electric field generating circuit 2120.

In some embodiments, various components within medical device 2100 can include a therapy output circuit, for example, an electric field sensing circuit 2122 configured to generate a signal corresponding to sensed electric fields. Electric field sensing circuit 2122 can be integrated with control circuitry 2106 or it can be separate from control circuitry 2106.

Sensing electrodes can be disposed on or adjacent to the housing of the medical device, on one or more leads connected to the housing, on a separate device implanted near or in the tumor, or any combination of these locations. In some embodiments, the electric field sensing circuit 2122 can include a first sensing electrode 2132 and a second sensing electrode 2134. In other embodiments, the housing 102 itself can serve as a sensing electrode for the electric field sensing circuit 2122. The electrodes 2132 and 2134 can be in communication with the electric field sensing circuit 2122. The electric field sensing circuit 2122 can measure the electrical potential difference (voltage) between the first electrode 2132 and the second electrode 2134. In some embodiments, the electric field sensing circuit 2122 can measure the electrical potential difference (voltage) between the first electrode 2132 or second electrode 2134, and an electrode disposed along the length of one or more leads 106. In some embodiments, the electric field sensing circuit can be configured to measure sensed electric fields and to record electric field strength in V/cm.

It will be appreciated that the electric field sensing circuit 2122 can additionally measure an electrical potential difference between the first electrode 2132 or the second electrode 2134 and the housing 102 itself. In other embodiments, the medical device can include a third electrode 2136, which can be an electric field sensing electrode or an electric field generating electrode. In some embodiments, one or more sensing electrodes can be disposed along lead 106 and can serve as additional locations for sensing an electric field. Many combinations can be imagined for measuring electrical potential difference between electrodes disposed along the length of one or more leads 106 and the housing 102 in accordance with the embodiments herein.

In some embodiments, the one or more leads 106 can be in electrical communication with the electric field generating circuit 2120. The one or more leads 106 can include one or more electrodes 218, as shown in FIG. 2. In some embodiments, various electrical conductors, such as electrical conductors 2126 and 2128, can pass from the header 104 through a feed-through structure 2130 and into the interior volume 2102 of medical device 2100. As such, the electrical conductors 2126 and 2128 can serve to provide electrical communication between the one or more leads 106 and control circuitry 2106 disposed within the interior volume 2102 of the housing 102.

While FIG. 21 illustrates the header 104 receiving a single lead 106 and/or lead plug or pin, it will be appreciated that this is for ease of illustration and that the header 104 can be configured to receive a plurality of leads 106 and/or lead plugs/pins. In some embodiments, the header 104 can be configured to receive 1, 2, 3, 4, 5, 6, 7, 8, or more leads 106 and/or lead plugs/pins. In some embodiments, the header 104 can be configured to receive leads consistent with the DF-1, VS-1, IS-1, LV-1 and IS-4 standards, amongst other standards.

In some embodiments, recorder circuitry can be configured to record the data produced by the electric field sensing circuit 2122 and record time stamps regarding the same. In some embodiments, the control circuitry 2106 can be hardwired to execute various functions, while in other embodiments the control circuitry 2106 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

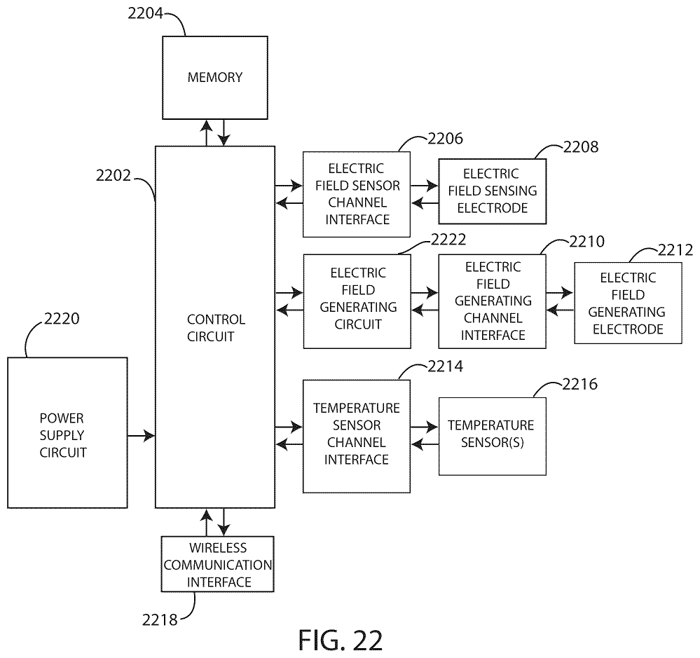

Elements of various embodiments of the medical devices described herein are shown in FIG. 22. However, it will be appreciated that some embodiments can include additional elements beyond those shown in FIG. 22. In addition, some embodiments may lack some elements shown in FIG. 22. The medical devices as embodied herein can gather information through one or more sensing channels and can output information through one or more field generating channels. A microprocessor 2202 can communicate with a memory 2204 via a bidirectional data bus. The memory 2204 can include read only memory (ROM) or random-access memory (RAM) for program storage and RAM for data storage. The microprocessor 2202 can also be connected to a wireless communication interface 2218 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 2220. In some embodiments, the medical device can include an inductive energy receiver coil interface (not shown) communicatively coupled or attached thereto to facilitate transcutaneous recharging of the medical device.

The medical device can include one or more electric field sensing electrodes 2208 and one or more electric field sensor channel interfaces 2206 that can communicate with a port of microprocessor 2202. The medical device can also include one or more electric field generating circuits 2222, one or more electric field generating electrodes 2212, and one or more electric field generating channel interfaces 2210 that can communicate with a port of microprocessor 2202. The medical device can also include one or more temperature sensors 2216 and one or more temperature sensor channel interfaces 2214 that can communicate with a port of microprocessor 2202. The channel interfaces 2206, 2210, and 2214 can include various components such as analog-to-digital converters for digitizing signal inputs, sensing amplifiers, registers which can be written to by the control circuitry to adjust the gain and threshold values for the sensing amplifiers, source drivers, modulators, demodulators, multiplexers, and the like.

In some embodiments, one or more physiological sensors can also be included herein. In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the temperature sensors 2216 are shown as part of a medical device in FIG. 22, it is realized that in some embodiments one or more of the sensors could be physically separate from the medical device. In various embodiments, one or more of the can be within another implanted medical device communicatively coupled to a medical device via wireless communication interface 2218. In yet other embodiments, one or more of the sensors can be external to the body and coupled to a medical device via wireless communication interface 2218.

### Electric Field Therapy Parameters

In some embodiments, medical devices herein can generate one or more electric fields at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation.

In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 1 MHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 100 mAmp, 125 mAmp, 150 mAmp, 175 mAmp, 200 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm. In some embodiments, the system can be configured to generate one or more applied electric field strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10 V/cm, 20 V/cm, 30 V/cm, 40 V/cm, 50 V/cm, 60 V/cm, 70 V/cm, 80 V/cm, 90 V/cm, 100 V/cm, 125 V/cm, 150 V/cm, 175 V/cm, 200 V/cm, 225 V/cm, 250 V/cm, 275 V/cm, 300 V/cm, 325 V/cm, 350 V/cm, 375 V/cm, 400 V/cm, 425 V/cm, 450 V/cm, 475 V/cm, 500 V/cm, 600 V/cm, 700 V/cm, 800 V/cm, 900 V/cm, 1000 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

In some embodiments, the electric field can be modulated in response to a patient's measured reference or core body temperature and/or a set period of time elapsing. For example, if the patient's reference or core body temperature is higher than a threshold level, the therapy parameters can be modulated to reduce the heat output of the system inside the body. If the patient's reference or core body temperature is higher than a threshold level, the electric field strength can be decreased by 5%, 10%, 15%, 20%, 25%, 30%, 35%, 40%, 45%, 50%, 55%, 60%, 65%, 70%, 75%, 80%, 85%, 90%, 95%, or 100% (e.g., turned off) or can be decreased by an amount falling within a range between any of the foregoing. If the patient's reference or core body temperature is higher than a threshold level, the electric field strength can be decreased by from 5% to 100%, or between 5% and 95%. It will be appreciated that other parameters can also be modulated in order to reduce the amount of heat generated by the system including, for example, current, voltage, and/or frequency.

Alternatively, if the reference or core body temperature is lower than a threshold level, then to maximize exposure to therapeutic electrical fields the electric field strength can be increased. In some embodiments, the electric field strength can be increased by 5%, 10%, 15%, 20%, 25%, 50%, 75%, 100%, 200%, 300%, 500%, 1000% or more, or by an amount falling within a range between any of the foregoing. It will be appreciated that other parameters can also be modulated in order to increase the intensity of the electrical field therapy provided by the system including, for example, current, voltage, and/or frequency.

In some embodiments, the electrical field therapy can be applied as part of a duty cycle scheme. The duty cycle with respect to any given electrode pair or across all electrodes can vary from 100% (continuous) to 90, 80, 70, 60, 50, 40, 30, 20, 10, 5 percent or less. In some embodiments, the implantable cancer treatment system can be configured to execute a duty cycle scheme alternating between different electrodes and/or different discrete segments within an electrode and/or electrode zone. For example, in some embodiments electrical fields can be delivered using one or more electrodes and/or individual discrete segments during a first phase of a cycle alternating with a separate electrode and/or one or more individual discrete segments during a second phase of the cycle. In some embodiments, the duty cycle scheme can be modulated (controlled or changed). For example, in some embodiments the duty cycle scheme can be modulated based on a signal from a temperature sensor, such as reducing, increasing, or otherwise changing the duty cycle scheme when the temperature crosses one or more threshold values.

In some embodiments, the medical device can have multiple phases in a therapy cycle. In various embodiments, different phases can include the use of different combinations of electrodes for generating electrical fields. As an example a first phase can include generating an electrical field utilizing a first electrode and a second electrode serving as poles, a second phase can include utilizing the first electrode and a third electrode, and a third phase can include utilizing the second electrode and a fourth electrode. Various combinations of electrodes for different phases are possible. Further some phases can include combinations of electrodes (serving as a positive or negative pole) paired with one or more other electrodes (serving as the other pole). As an example, a phase could include the first electrode in combination with the second electrode wherein the combination is paired with the third electrode.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable lead for a cancer treatment system comprising:
a lead body, the lead body comprising
a proximal end; and
a distal end;
one or more electric field generating electrodes, wherein the electrodes are disposed along a length of the lead body;
a plurality of electrical wires;
wherein the plurality of electrical wires are disposed within the lead body;
wherein the plurality of electrical wires provide an electrical connection between at least one of the electrodes and the proximal end of the lead body; and
wherein at least two of the plurality of electrical wires are arranged in parallel and connected to the same electrode.

2. The implantable lead for a cancer treatment system of claim 1, the electrodes comprising:
a proximal end; and
a distal end; and
wherein the plurality of electrical wires arranged in parallel are connected to a middle portion of the electrodes between the proximal end and the distal end.

3. The implantable lead for a cancer treatment system of claim 1, wherein a set of the plurality of electrical wires reflecting all wires connected to a particular electrode of the one or more electric field generating electrodes have a resistance of less than 0.4 ohm/cm.

4. The implantable lead for a cancer treatment system of claim 1, wherein a set of the plurality of electrical wires reflecting all wires connected to a particular electrode of the one or more electric field generating electrodes have a resistance of less than or equal to 0.2 ohm/cm.

5. The implantable lead for a cancer treatment system of claim 1, further comprising:
a connection plug, the connection plug comprising a plurality of electrical terminals;
wherein the connection plug is disposed at the proximal end of the lead body; and
wherein the connection plug is in electrical communication with the plurality of electrical wires.

6. The implantable lead for a cancer treatment system of claim 5, wherein the resistance of the electrical wires arranged in parallel and connected to the same electrode as measured from an electrical terminal to a point of connection with the electrode is less than 5 ohms.

7. The implantable lead for a cancer treatment system of claim 5, wherein the resistance of the electrical wires arranged in parallel and connected to the same electrode as measured from an electrical terminal to a point of connection with the electrode is less than or equal to 3.5 ohms.

8. The implantable lead for a cancer treatment system of claim 1, wherein the electrodes are disposed circumferentially around the lead body and extend a distance along a longitudinal axis of the lead body.

9. The implantable lead for a cancer treatment system of claim 1, wherein at least three of the plurality of electrical wires are arranged in parallel and connected to the same electrode.

10. The implantable lead for a cancer treatment system of claim 1, wherein the plurality of electrical wires have a diameter of less than 130 microns.

11. The implantable lead for a cancer treatment system of claim 1, the electrodes comprising a flat ribbon of metal, wherein the flat ribbon of metal is wrapped around the lead body.

12. The implantable lead for a cancer treatment system of claim 1, the electrodes comprising at least two electrical field generating electrodes, wherein the at least two electrical field generating electrodes are separated by a distance along a longitudinal axis of the lead body.

13. An implantable lead for a cancer treatment system comprising:
a lead body, the lead body comprising
a proximal end; and
a distal end;
one or more electric field generating electrodes, the electrodes comprising a plurality of discrete segments, wherein the plurality of discrete segments are electrically isolated from direct connection with one another;
wherein the electrodes are disposed along a length of the lead body;
a plurality of electrical wires;
wherein the plurality of electrical wires are disposed within the lead body;
wherein the plurality of electrical wires provide an electrical connection between the electrodes and the proximal end of the lead body; and
wherein the plurality of electrical wires are separately connected to the discrete segments of the electrodes.

14. The implantable lead for a cancer treatment system of claim 13, wherein the plurality of discrete segments have a length along the longitudinal axis of the lead body from 0.25 to 70 mm.

15. The implantable lead for a cancer treatment system of claim 13, wherein the plurality of discrete segments wrap around the lead body circumferentially.

16. The implantable lead for a cancer treatment system of claim 13, further comprising a temperature sensor, wherein the temperature sensor is disposed between two adjacent discrete segments.

17. An implantable cancer treatment system comprising:
an implantable housing, the implantable housing comprising
an electric field generating circuit; and
control circuitry;
wherein the control circuitry causes the electric field generating circuit to generate the one or more electric fields at frequencies selected from a range of between 10 kHz to 1 MHz within a bodily tissue;
wherein the one or more electric fields are effective to prevent and/or disrupt cellular mitosis in a cancerous cell;
a therapy lead, the therapy lead comprising
a lead body, the lead body comprising
a proximal end; and
a distal end;
one or more electric field generating electrodes, the electrodes comprising a plurality of discrete segments, wherein the plurality of discrete segments are electrically isolated from direct connection with one another;
wherein the electrodes are disposed along a length of the lead body;
a plurality of electrical wires;
wherein the plurality of electrical wires are disposed within the lead body;
wherein the plurality of electrical wires provide an electrical connection between the electrodes and the proximal end of the lead body;
wherein the plurality of electrical wires are separately connected to the discrete segments of the electrodes; and
wherein the implantable cancer treatment system is configured to execute a duty cycle scheme wherein electrical fields are delivered using one or more individual discrete segments during a first phase of a cycle alternating with a separate one or more individual discrete segments during a second phase of the cycle.

18. The implantable cancer treatment system of claim 17, wherein the plurality of discrete segments have a length along the longitudinal axis of the lead body from 0.25 to 70 mm.

19. The implantable cancer treatment system of claim 17, the therapy lead further comprising a temperature sensor, wherein the temperature sensor is disposed between two adjacent discrete segments.

20. The implantable cancer treatment system of claim 19, wherein the duty cycle scheme is modulated based on a signal from the temperature sensor.
